(19) United States
(12) Patent Application Publication
(10) Pub. No.: US 2021/0293811 A1
(43) Pub. Date: Sep. 23, 2021

(54) TARGET INTERFERENCE MITIGATION IN ANTI-DRUG ANTIBODY ASSAY

(71) Applicant: Regeneron Pharmaceuticals, Inc., Tarrytown, NY (US)

(72) Inventors: Jihua Chen, Ossining, NY (US); Kimberly Kendra, Carmel, NY (US); Albert Torri, LaGrangeville, NY (US); Giane Oliveira Sumner, Mahwah, NJ (US)

(21) Appl. No.: 17/203,566

(22) Filed: Mar. 16, 2021

Related U.S. Application Data
(60) Provisional application No. 62/990,681, filed on Mar. 17, 2020.

(51) Int. Cl. G01N 33/563; G01N 33/543

(52) U.S. Cl. CPC G01N 33/563; G01N 33/54306

(57) ABSTRACT

The present invention provides methods and systems to detect, quantify or characterize anti-drug antibodies which are induced by the administration of pharmaceutical products. The methods and systems include the use of a binding partner of a target and/or a co-factor to improve the detection of anti-drug antibodies in serum samples in the presence of soluble targets based on competitive target binding. The methods and systems also include the use of immuno-depletion to improve the detection.

### FIELD

The present invention generally pertains to methods and systems to characterize, identify and/or measure anti-drug antibodies which are induced by the administration of pharmaceutical products. These methods and systems are based on competitive ligand binding.

### BACKGROUND

There are concerns of drug efficacy and patient safety due to the presence of antibodies which are induced by the administration of pharmaceutical products, for example, the induction of anti-drug antibodies (ADAs), since the ADAs can contribute to some clinical consequences, such as reducing drug efficacy, cross-reacting to endogenous proteins, or altering the pharmacokinetics of therapeutic proteins. FDA recommends adoption of a risk-based approach for evaluating and mitigating immune responses regarding adverse immunologically related responses associated with therapeutic protein products that affect their safety and efficacy. (Guidance for Industry: Immunogenicity Assessment for Therapeutic Protein Products, August 2014, U.S. Department of Health and Human Services, Food and Drug Administration)

The prescribing information and FDA's clinical pharmacology review of 121 FDA approved biologics were reviewed for evaluating and reporting immunogenicity data, including monoclonal antibodies, enzyme products, cytokines, growth factors and toxins. The highest frequency of reporting was for immunogenicity incidences. The clinical significance of ADAs was unknown. Overall, there was a striking concordance between an increase in systemic clearance of products and a reduction of efficacy associated with ADAs. (Wang et al., Evaluating and Reporting the Immunogenicity Impacts for Biological Products-a Clinical Pharmacology Perspective. The AAPS Journal. 2016; 18(2): 395-403)

The biological complexity of immune responses presents challenges in evaluating the impact of ADAs on pharmacokinetics, since pharmacokinetic exposure can be more sensitive than efficacy endpoints for evaluating ADA effects. It will be appreciated that a need exists for methods and systems to characterize, identify and/or measure ADAs, such as to improve the methods and systems of ADA detection. These methods and systems can provide valuable information regarding immunogenicity impacts in clinical pharmacology relevant to pharmacokinetics, efficacy, and safety for drug administrations, such as the administration of biologicals.

### SUMMARY

Biologics, such as monoclonal antibodies, are therapeutic proteins with clinical applications across a wide range of conditions, such as cancer, cardiovascular disease, infectious disease or autoimmune disorders. The immunogenicity incidences of protein pharmaceutical products have led to an increasing demand for characterizing the presence of antibodies which are induced by the administration of protein pharmaceutical products, for example, anti-drug antibodies (ADAs). The characterization and measurement data of ADAs can provide the understanding of immunogenicity of pharmaceutical products for enhancing drug safety.

Exemplary embodiments disclosed herein satisfy the aforementioned demands by providing methods and systems for characterizing, identifying and/or measuring ADAs which are induced by the administration of pharmaceutical products. This disclosure provides a method of identifying an anti-drug antibody in a sample, comprising: contacting the sample with a first labeled drug, contacting the sample with a second labeled drug, contacting the sample with a binding partner of a target, and detecting the presence of a complex which comprises the first labeled drug, the anti-drug antibody and the second labeled drug; wherein the sample comprises the anti-drug antibody and the target, and wherein the target is a binding partner of the drug.

In some exemplary embodiments, the method of identifying an anti-drug antibody in a sample further comprises contacting the sample with a co-factor to enhance the binding between the target and the binding partner of the target. In some aspects, the method of identifying an anti-drug antibody in a sample is conducted under a mild acidic assay pH.

In some aspects, the method of identifying an anti-drug antibody in a sample further comprises removing the target using an anti-target antibody, wherein the anti-target antibody is attached to a solid support.

In some aspects, the first labeled drug or the second labeled drug of the method is ruthenium labeled drug or biotinylated drug. In some aspects, the binding partner of the target of the method is a natural binding partner or a receptor of the target, wherein the target is a soluble multimeric target.

In some aspects, the mild acidic assay pH of the method is in the range of from about pH 4.5 to about pH 6.5, is about pH 6.0 or is about pH 5.0.

In some aspects, the drug of the method is a chemical compound, a nucleic acid, a toxin, a peptide, a protein, a fusion protein, an antibody, an antibody fragment, a Fab region of an antibody, an antibody-drug conjugate, or a pharmaceutical product. In some aspects, the drug of the method is an antibody and the sample is a serum sample.

This disclosure, at least in part, provides a system for identifying an anti-drug antibody in a sample, comprising: a first labeled drug, a second labeled drug, a binding partner of a target, and an assay system to detect the presence of a complex which comprises the first labeled drug, the anti-drug antibody and the second labeled drug; wherein the sample comprises the anti-drug antibody and the target, and wherein the target is a binding partner of the drug.

In some exemplary embodiments, the system further comprises a co-factor which can enhance the binding between the target and the binding partner of the target. In some aspects, the sample of the system is treated with a solution having a mild acidic assay pH. In some aspects, the system further comprises an anti-target antibody, wherein the anti-target antibody is attached to a solid support.

In some aspects, the first labeled drug or the second labeled drug of the system is ruthenium labeled drug or biotinylated drug. In some other aspects, the binding partner of the target of the system is a natural binding partner or a receptor of the target, wherein the target is a soluble multimeric target.

In some aspects, the mild acidic assay pH of the system is in the range of from about pH 4.5 to about pH 6.5, is about pH 6.0 or is about pH 5.0.

In some aspects, the drug of the system is a chemical compound, a nucleic acid, a toxin, a peptide, a protein, a fusion protein, an antibody, an antibody fragment, a Fab region of an antibody, an antibody-drug conjugate, or a pharmaceutical product. In other aspects, the drug of the system is an antibody and the sample of the system is a serum sample.

These, and other, aspects of the invention will be better appreciated and understood when considered in conjunction with the following description and the accompanying drawings. The following description, while indicating various embodiments and numerous specific details thereof, can be given by way of illustration and not of limitation. Many substitutions, modifications, additions, or rearrangements may be made within the scope of the invention.

### BRIEF DESCRIPTION OF THE DRAWINGS

FIG. 1 shows the presence of target-mediated signals due to the presence of soluble multimeric target in serum samples, since the multimeric target protein can bind to ruthenium labeled drug and biotinylated drug simultaneously under neutral assay pH for conducting bridging ADA assays according to an exemplary embodiment. The incorporation of natural binding partner of the drug target and a co-factor to the bridging ADA assay under mild acidic assay pH can mitigate the target-mediated signals according to an exemplary embodiments.

FIG. 2A shows the screening of several anti-target antibodies, for example, Ab1-Ab9, at 100 μg/mL in comparing to control (Ctrl) for mitigating target interferences in monkey naïve serum sample according to an exemplary embodiment.

FIG. 2B shows the use of a commercially available polyclonal anti-target antibody to mitigate target interference in monkey naïve serum sample according to an exemplary embodiment.

FIG. 3 shows the incorporation of target receptor to the bridging ADA assay to improve ADA detection by mitigating target-mediated signals according to an exemplary embodiment.

FIG. 4A shows the incorporation of target receptor and co-factor to the bridging ADA assay to improve ADA detection by mitigating target-mediated signals according to an exemplary embodiment. Different concentrations of the co-factor protein were added to the solution containing 50 μg/mL of the soluble target receptor for conducting bridging ADA assay according to an exemplary embodiment.

FIG. 4B shows that a widely variable range of target-mediated assay signals were detected in the absence of any blocker proteins in eight naïve monkey serum samples (control) according to an exemplary embodiment. The presence of target receptor (50 μg/mL) and co-factor (50 μg/mL) showed effective mitigation of the target-mediated assay signals in all monkey serum samples according to an exemplary embodiment.

FIG. 5 shows the optimization of assay pH to mitigate target interference in bridging ADA assays using four experimental designs according to an exemplary embodiment. The four experimental designs were (1) four monkey naïve serum samples at neutral pH (control); (2) four monkey naïve serum samples with 50 μg/mL of the receptor and 50 μg/mL of the co-factor at neutral pH; (3) four monkey naïve serum samples at mild acidic pH (at about pH 6.0); and (4) four monkey naïve serum samples with 50 μg/mL of the receptor and 50 μg/mL of the co-factor at about pH 6.0 according to an exemplary embodiment.

FIG. 6A shows the determination of the target tolerance level using a recombinant target protein under different assay pH conditions, when the ADA assay was performed using 50 μg/mL of both the receptor and co-factor proteins according to an exemplary embodiment.

FIG. 6B shows the detection of ADA signals under different assay pH conditions using early bleeds from MAB-Y Fab-immunized rabbits according to an exemplary embodiment.

FIG. 7A shows the drug concentrations in serum samples of two monkeys which were administrated with a single dose of drug, for example, MAB-Y, according to an exemplary embodiment. LLOQ indicates lower limit of quantitation.

FIG. 7B shows target concentrations and ADA signals in Day 0, 28 and 52 samples with different assay conditions including the incorporation of target receptor, co-factor and mild acidic assay pH to bridging ADA assay to improve ADA detection using monkey post-dose samples according to an exemplary embodiment.

FIG. 8 shows the drug concentrations in serum samples of three subjects with a single dose of MAB-Y according to an exemplary embodiment. LLOQ indicates lower limit of quantitation.

FIG. 9 shows target concentrations and ADA signals in Day 0, 29 and 64 samples with different assay conditions including the incorporation of target receptor, co-factor and mild acidic assay pH to bridging ADA assay to improve ADA detection according to an exemplary embodiment.

FIG. 10A shows immuno-depletion of the target protein with MAB-A conjugated magnetic beads at neutral assay pH to eliminate target-mediated signal in drug-free naïve human serum samples according to an exemplary embodiment.

FIG. 10B shows immuno-depletion of the target protein with MAB-A conjugated magnetic beads at neutral assay pH to eliminate target-mediated signal in in baseline serum samples according to an exemplary embodiment.

FIG. 11 shows ADA assay signals in Day 1, 15, 29 and 57 samples from four monkeys were measured under different assay conditions, for example, without blockers under neutral assay pH, with 100 μg/mL MAB-A under neutral assay pH, without blockers under mild acidic pH (pH ˜6.0), and with 100 μg/mL MAB-A under mild acidic pH (pH ˜6.0) according to an exemplary embodiment.

FIG. 12 shows target concentrations and ADA assay signal in Day 1, 15, 29, and 57 samples before and after immuno-depletion with MAB-A conjugated magnetic beads under mild acidic assay pH according to an exemplary embodiment.

FIG. 13 shows immuno-depletion using MAB-A conjugated magnetic beads in baseline samples and post-dose samples under different pH conditions according to an exemplary embodiment.

FIG. 14 shows the detection of true ADA signals in Day 1, 15, 29 and 57 samples from an ADA-positive subject using the competitive blocker ADA method including soluble receptor (50 μg/mL) and co-factor (50 μg/mL) under mild acidic assay pH and the modified immuno-depletion method according to an exemplary embodiment.

### DETAILED DESCRIPTION

The increasing concerns of drug efficacy and patient safety due to immunogenicity incidences of protein pharmaceutical products have led to an increasing demand for characterizing the anti-drug antibodies (ADAs). The demands of characterizing ADAs are driven by, for example, the needs of understanding the impacts of ADAs on reducing the drug efficacy, cross-reacting to endogenous proteins, or altering the pharmacokinetics of pharmaceutical products. The characterization data of ADAs can provide valuable information regarding immunogenicity of pharmaceutical products, and therefore to enhance the safety for drug administrations.

The administration of biologics, such as monoclonal antibodies, can induce immune responses in animal subjects and human patients, such as the development of anti-drug antibodies (ADAs). The immunogenicity responses induced by therapeutic proteins can range from transient ADAs with no clinical significance to the generation of high titer, persistent ADAs which may lead to reduced drug exposure, lack or loss of efficacy and adverse events, such as hypersensitivity reaction, anaphylaxis and injection site reactions (Koren et al., Recommendations on risk-based strategies for detection and characterization of antibodies against biotechnology products. J Immunol Methods, 2008. 333(1-2): p. 1-9). There were reported incidences concerning the neutralizing activity of ADAs, such as immunogenicity impacts in clinical pharmacology relevant to pharmacokinetics, efficacy, and safety. The formation of ADAs during drug treatment may cause a decrease in drug concentration in patient's body, which may contribute to the reduced efficacy. Various ADAs which are capable of binding to different sites of the drugs can be present in patient's bodies, such as neutralizing or non-neutralizing ADAs. Neutralizing ADAs are capable of binding to the active site of the drug molecule, such as the binding site in drug molecule for binding to the drug target, or the variable regions of an antibody drug. When the neutralizing ADA binds to the active site of a drug, it renders the drug becoming inactive. The non-neutralizing ADA can be capable of binding to the non-active site of the drug molecule, such as the constant region or the scaffold of an antibody drug molecule. Even though the drug can be still active subjected to the binding of the non-neutralizing ADAs, the presence of non-neutralizing ADAs may contribute to certain changes in clinical pharmacology.

Immunogenicity refers to the propensity of the therapeutic product to generate immune responses to itself and to related proteins, such as inducing immunologically related adverse clinical events. Relevant immunogenicity information includes the induction of binding antibodies, the induction of neutralizing antibodies, altered pharmacokinetics, reduced efficacy, and safety concerns. However, the clinical significance of ADAs was unknown. In addition, the limited available data may preclude a determination of the effect of ADAs. ADAs may associate with a concordance between an increase in systemic clearance of pharmaceutical products and a reduction of efficacy. Some drug products had drug-sustaining ADAs which resulted in a reduced clearance possibly due to the formation of ADA-drug complex, such as ADA binding of the drug. (Wang et al., Evaluating and Reporting the Immunogenicity Impacts for Biological Products-a Clinical Pharmacology Perspective. The AAPS Journal. 2016; 18(2): 395-403)

Therefore, immunogenicity assessment may be required by regulatory agencies as part of product safety, and the incidence of ADA and neutralizing antibody (NAb) are part of the prescribing information (US Department of Health and Human Services, U.F.C., CBER, Guidance for Industry—Assay Development for Immunogenicity Testing of Therapeutic Proteins (Draft). US Department of Health and Human Services, Washington, D.C., USA, 2009; European Medicines Agency, C.f.M.P.f.H.U., Guideline on Immunogenicity Assessment of Biotechnology-Drived Therapeutic Proteins. European Medicines Agency, London, U K, 2007). Thus, the accurate detection of ADA is an important aspect of any biological drug development programs (Mire-Sluis, et al., Recommendations for the design and optimization of immunoassays used in the detection of host antibodies against biotechnology products. J Immunol Methods, 2004. 289(1-2): p. 1-16; Shankar, G., et al., Recommendations for the validation of immunoassays used for detection of host antibodies against biotechnology products. J Pharm Biomed Anal, 2008. 48(5): p. 1267-81).

The assays for identifying or measuring ADAs are usually bridging immunoassays by incorporating biotinylated drug (Bio-drug) and ruthenium labeled drug (Ru-drug) as the bridging components, for example, the formation of an ADA-drug complex comprising biotinylated drug, ADA and ruthenium labeled drug. The bridging ADA assays provide high throughput and sensitivity, as well as the ability to detect most ADA antibody isotypes. However, the bridging ADA assay can be susceptible to several interferences. The presence of certain molecules in samples can cause the interferences, such as the presence of free drugs, soluble drug targets and other matrix proteins in serum samples. The soluble drug targets can be dimeric or multimeric peptides or proteins. In particular, it is challenging to overcome the interferences contributed by the soluble dimeric or multimeric target due to the highly specific binding between the target and the drug. It is even more challenging in considering the highly variable biologic properties of each target protein. (Zhong, et al., Drug Target Interference in Immunogenicity Assays: Recommendations and Mitigation Strategies. AAPS J, 2017. 19(6): p. 1564-1575.)

The incorporation of target-specific antibodies in bridging ADA assays was used to mitigate target interference by blocking the interfering signals (Liao, K., et al., Inhibition of interleukin-5 induced false positive anti-drug antibody responses against mepolizumab through the use of a competitive blocking antibody. J Immunol Methods, 2017. 441: p. 15-23.; Zhong, et al., Identification and inhibition of drug target interference in immunogenicity assays. J Immunol Methods, et al., 2010. 355(1-2): p. 21-8.). However, a suitable blocking antibody may not be readily available for many monoclonal antibody drugs. A suitable blocking antibody should be able to competitively bind to the target without affecting the bridging interactions between ADA and labeled drug molecules. Other strategies of mitigating target interferences include the use of target binding proteins, target immuno-depletion and certain type of lectins to inhibit the interference from the heavily glycosylated target proteins (Carrasco-Triguero, et al., Overcoming soluble target interference in an anti-therapeutic antibody screening assay for an antibody-drug conjugate therapeutic. Bioanalysis, 2012. 4(16): p. 2013-26.).

Adjustments of the pH conditions of the bridging ADA assays through acid pretreatment or sample incubation under mild acidic or basic pH conditions are additional strategies to disrupt or enhance various interactions contributed by drug, ADA, drug target or labeled drug molecules. The specific pH conditions for each individual ADA assay should be carefully evaluated, since changes in pH conditions can also lead to unintended consequences, such as releases of free drugs from target-drug complexes or dimerization of monomeric drug targets, which can increase false-positive signals. (Dai, et al., Development of a method that eliminates false-positive results due to nerve growth factor interference in the assessment of fulranumab immunogenicity. AAPS J, 2014. 16(3): p. 464-77; Zoghbi, et al., A breakthrough novel method to resolve the drug and target interference problem in immunogenicity assays. J Immunol Methods, 2015. 426: p. 62-9)

The presence of drug target in study samples can pose a challenge for the development of reliable bridging ADA assays because drug targets, particularly dimeric or multimeric target proteins, can form bridging complexes between capture and detection molecules, giving rise to false-positive ADA signal. The addition of either an individual anti-target antibody or a cocktail of such antibodies is a common approach to mitigating target interference signal. These antibodies usually provide high specificity and target affinity and are relatively easy to produce in large quantities. However, anti-target antibodies must meet certain criteria to be effective. To avoid cross-reactivity between these antibodies and ADAs, the anti-target antibodies should not share similar or overlapping CDR or framework sequences with the drug molecule. In addition, blocker antibodies should not contain a human IgG constant region sequence, as these sequences may compete with ADA detection of similar sequences on labeled drug molecules, resulting in false negative ADA results.

This disclosure provides methods and systems to satisfy the aforementioned requirements by providing methods and systems for characterizing, identifying and measuring ADAs which are induced by the administration of pharmaceutical products. In particular, the methods and systems of the present application provide an improvement to mitigate the target interferences by incorporating a natural binding partner of the drug target to the bridging ADA assay, such as a receptor of the drug target. In some exemplary embodiments, a binding co-factor of the drug target is incorporated to the bridging ADA assay to mitigate the target interferences, wherein the binding co-factor of the drug target can facilitate the binding between the drug target and the natural binding partner. In some aspects, the natural binding partner of the drug target is a target receptor which has a high affinity to the drug target and can compete with the drug for target binding. In some aspects, the target receptor and the co-factor are incorporated to the bridging ADA assay to improve ADA detection. As the natural binding partner of the target, the receptor possesses a high affinity to the target and can out-compete the drug for target binding. Endogenously, co-factor molecules help to maintain the structure of many receptor proteins and improve target-receptor binding.

The present application provides target-binding proteins, such as the soluble target receptor, with or without their requisite co-factor(s), for the inhibition of target interference. These proteins are the natural binding partners of the target and usually exhibit high target affinity. Based on the glycosylation characteristics of the soluble target protein and the glycan-binding specificity of lectins, certain lectins can also be used to mitigate target interference from the highly glycosylated target proteins (Carrasco-Triguero, M., et al., Overcoming soluble target interference in an anti-therapeutic antibody screening assay for an antibody-drug conjugate therapeutic. Bioanalysis, 2012. 4(16): p. 2013-26).

The present application also provides the strategy of altering the assay pH to mitigate target interference, by either directly affecting the dimeric or multimeric target protein formation or by changing the drug binding affinity to the target. The present application provides that mild acidic assay pH alone can at least partially mitigate the target-mediated signal probably by reducing the binding of target to the labeled drugs.

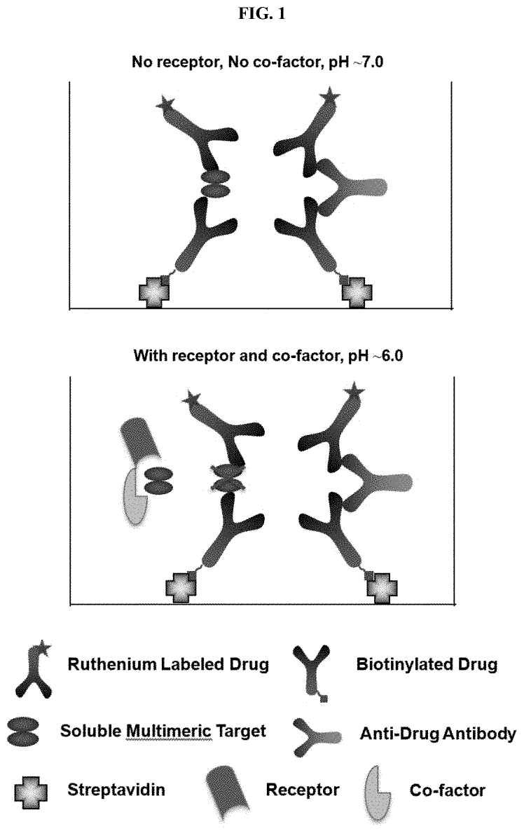

In one aspect, this disclosure provides methods and systems to mitigate the target interferences by incorporating a natural binding partner of the drug target and a co-factor to the bridging ADA assay under mild acidic assay pH. In some aspects, in the presence of the receptor and the co-factor proteins under mild acidic assay pH for conducting bridging ADA assays, the drug target can no longer bridge the labeled drugs (e.g., ruthenium labeled drug and biotinylated drug) due to the presence of two different activities, since these activities are synergistic. For example, as shown in FIG. 1, due to the presence of soluble multimeric target in serum samples, the multimeric target protein can bind to ruthenium labeled drug and biotinylated drug simultaneously (such as Bio-MAB-Y and Ru-MAB-Y) under neutral assay pH for conducting bridging ADA assays, which can generate target-mediated signals. In the presence of target receptor and co-factor proteins under mild acidic assay pH for conducting bridging ADA assays, the target-mediated signals can be mitigated, since the drug target can form a complex with drug receptor and co-factor as shown in FIG. 1. The use of mild acidic assay pH for conducting bridging ADA assays provides the advantages of reducing the binding between available drug targets and the labeled drugs. These competitive blockers, for example, receptor and/or co-factor, synergistically inhibit target interference and increase target tolerance levels, especially when the assay is performed under mild acidic conditions.

In some exemplary embodiments, a drug target is a multimeric protein which is present in serum, the drug target can generate target-mediated false-positive signal which can interfere ADA quantitation. For example, ADAs of MAB-Y (e.g., a drug) in serum samples can be detected using Ru-MAB-Y (ruthenium labeled MAB-Y) and Bio-MAB-Y (biotinylated MAB-Y) by forming a complex comprising Ru-MAB-Y, ADA and Bio-MAB-Y, for example, using ADA to bridge Ru-MAB-Y and Bio-MAB-Y. However, since the target of MAB-Y is a multimeric protein which is expressed at different levels in monkey and human naïve serum samples, the target in serum can form a complex with Ru-MAB-Y and Bio-MAB-Y, for example, using target to bridge Ru-MAB-Y and Bio-MAB-Y, which contribute to target-mediated false-positive signal. In some exemplary embodiments, an anti-target antibody is used to mitigate the interference of false-positive signal caused by the bridging effects of the drug target by removing the drug target through immuno-depletion. The immuno-depletion of target protein can be conducted using magnetic beads conjugated with an anti-target antibody, which is effective at mitigating target-mediated signal in combination with mild acidic assay pH. These methods allow detection of a true ADA signal in monkey and human post-dose serum samples.

The present application provides two different approaches to mitigate multimeric target interference in monkey and human serum samples including competition for target binding by soluble target receptor and co-factor proteins and immuno-depletion using anti-target antibody-conjugated magnetic beads. For both approaches, mild acidic assay conditions (such as pH ˜6.0) can selectively inhibit target binding to the labeled drug molecules or potentiate the binding of target to anti-target antibody. The combination of target receptor and co-factor proteins under a mild acidic assay pH can significantly reduce target-mediated signals in post-dose monkey serum samples and human clinical study samples to background levels. Immuno-depletion with mild acidic assay conditions can provide a greater than 50-fold reduction in target levels and eliminate target interference in clinical study samples while maintaining true positive ADA detection.

In one aspect, methods and systems are provided for characterizing, identifying and/or measuring an anti-drug antibody in a sample. They satisfy the long felt needs for characterizing the antibodies induced by the administration of drugs or pharmaceutical products, which can be used to study preclinical or clinical toxicology and pharmacokinetics. These methods and systems can be applied in preclinical toxicology or pharmacokinetic studies to monitor ADAs over time after the administration of the pharmaceutical products.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

As used herein, the terms “include,” “includes,” and “including,” are meant to be non-limiting and are understood to mean “comprise,” “comprises,” and “comprising,” respectively.

In some exemplary embodiments, this disclosure provides a method of identifying an anti-drug antibody in a sample, comprising: contacting the sample with a first labeled drug, contacting the sample with a second labeled drug, contacting the sample with a binding partner of a target, and detecting the presence of a complex which comprises the first labeled drug, the anti-drug antibody and the second labeled drug; wherein the sample comprises the anti-drug antibody and the target, and wherein the target is a binding partner of the drug. In some exemplary embodiments, the drug of the method is a chemical compound, a nucleic acid, a toxin, a peptide, a protein, a fusion protein, an antibody, an antibody fragment, a Fab region of an antibody, an antibody-drug conjugate, or a pharmaceutical product.

As used herein, the term “peptide” or “protein” includes any amino acid polymer having covalently linked amide bonds. Proteins comprise one or more amino acid polymer chains, generally known in the art as “peptide” or “polypeptides”. A protein may contain one or multiple polypeptides to form a single functioning biomolecule. In some exemplary embodiments, the protein can be an antibody, a bispecific antibody, a multispecific antibody, antibody fragment, monoclonal antibody, host-cell protein or combinations thereof.

As used herein, the term “pharmaceutical product” includes an active ingredient which can be fully or partially biological in nature or which has pharmaceutical activity. In some exemplary embodiments, the pharmaceutical product can comprise a drug, a peptide, a protein, a fusion protein, an antibody, an antibody fragment, a Fab region of an antibody, an antibody-drug conjugate, a peptide-drug conjugate, a Fc region of an antibody, an enzyme product, a cytokine, a growth factor, a pharmaceutical product, a toxin, a nucleic acid, DNA, RNA, a chemical compound, a cell, a tissue, an antigen, vaccine or any pharmaceutical ingredient which can be capable of inducing antibodies in a subject. In some other exemplary embodiments, the pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate or combinations thereof.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. An antibody fragment may be produced by various means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains that are linked together, for example, by disulfide linkages. An antibody fragment may optionally comprise a multi-molecular complex.

As used herein, the term “antibody-drug conjugate”, or “ADC” can refer to antibody attached to biologically active drug(s) by linker(s) with labile bond(s). An ADC can comprise several molecules of a biologically active drug (or the payload) which can be covalently linked to side chains of amino acid residues of an antibody (Siler Panowski et al., Site-specific antibody drug conjugates for cancer therapy, 6 mAbs 34-45 (2013)). An antibody used for an ADC can be capable of binding with sufficient affinity for selective accumulation and durable retention at a target site. Most ADCs can have Kd values in the nanomolar range. The payload can have potency in the nanomolar/picomolar range and can be capable of reaching intracellular concentrations achievable following distribution of the ADC into target tissue. Finally, the linker that forms the connection between the payload and the antibody can be capable of being sufficiently stable in circulation to take advantage of the pharmacokinetic properties of the antibody moiety (e.g., long half-life) and to allow the payload to remain attached to the antibody as it distributes into tissues, yet should allow for efficient release of the biologically active drug once the ADC can be taken up into target cells. The linker can be: those that are non-cleavable during cellular processing and those that are cleavable once the ADC has reached the target site. With non-cleavable linkers, the biologically active drug released within the call includes the payload and all elements of the linker still attached to an amino acid residue of the antibody, typically a lysine or cysteine residue, following complete proteolytic degradation of the ADC within the lysosome. Cleavable linkers are those whose structure includes a site of cleavage between the payload and the amino acid attachment site on the antibody. Cleavage mechanisms can include hydrolysis of acid-labile bonds in acidic intracellular compartments, enzymatic cleavage of amide or ester bonds by an intracellular protease or esterase, and reductive cleavage of disulfide bonds by the reducing environment inside cells.

As used herein, an “antibody” is intended to refer to immunoglobulin molecules consisting of four polypeptide chains, two heavy (H) chains and two light (L) chains inter-connected by disulfide bonds. Each heavy chain has a heavy chain variable region (HCVR or VH) and a heavy chain constant region. The heavy chain constant region contains three domains, CH1, CH2 and CH3. Each light chain has of a light chain variable region and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL can be composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” is inclusive of, but not limited to, those that are prepared, expressed, created or isolated by recombinant means, such as antibodies isolated from a host cell transfected to express the antibody. An IgG comprises a subset of antibodies.

### Exemplary Embodiments

Embodiments disclosed herein provide compositions, methods, and systems for identifying an anti-drug antibody in a sample.

In some exemplary embodiments, the disclosure provides a method of identifying an anti-drug antibody in a sample, comprising: contacting the sample with a first labeled drug, contacting the sample with a second labeled drug, contacting the sample with a binding partner of a target, and detecting the presence of a complex which comprises the first labeled drug, the anti-drug antibody and the second labeled drug; wherein the sample comprises the anti-drug antibody and the target, and wherein the target is a binding partner of the drug. In some aspects, the method of identifying an anti-drug antibody in a sample is conducted under a mild acidic assay pH.

In some aspects, the mild acidic assay pH of the method is in the range of about pH 4.5-6.5, about pH 3-6.9, about pH 4-6.5, about pH 4.5-6.5, about pH 5-6.5, about pH 5.5-6.5, about 5.9-6.2, about pH 5.0 or preferably about pH 6.0.

In some aspects, the method of identifying an anti-drug antibody in a sample further comprises removing the target using an anti-target antibody, wherein the anti-target antibody is attached to a solid support.

In some aspects, the solid support in the method or system of the present application can be beads, magnetic beads, chromatography resins, polymer, or chromatography matrix.

It is understood that the system is not limited to any of the aforesaid pharmaceutical products, peptides, proteins, antibodies, anti-drug antibodies, protein complexes, or pharmaceutical products.

The consecutive labeling of method steps as provided herein with numbers and/or letters is not meant to limit the method or any embodiments thereof to the particular indicated order. Various publications, including patents, patent applications, published patent applications, accession numbers, technical articles and scholarly articles are cited throughout the specification. Each of these cited references is herein incorporated by reference, in its entirety and for all purposes, herein. The disclosure will be more fully understood by reference to the following Examples, which are provided to describe the disclosure in greater detail. They are intended to illustrate and should not be construed as limiting the scope of the disclosure.

### EXAMPLES

### Reagent Preparations

The solutions which were used for the total drug assay and the total target assay were prepared in assay dilution buffer (ADB: 0.5% BSA, 0.05% Tween-20, 1×PBS). The solutions which were used in the ADA assay were prepared in 1% BSA, 1×PBS. (ADB is assay dilution buffer; BSA is bovine serum albumin; PBS is phosphate-buffered saline) PBS was purchased from Gibco (Grand Island, N.Y.). 1.5 M Trizma base was purchased from Sigma (St Louis, Mo.). Glacial acetic acid was purchased from Thermo Fisher Scientific (Waltham, Mass.). Monkey and human serum were purchased from Bioreclamation (Westbury, N.Y.). Streptavidin-coated microplates were purchased from Meso Scale Discovery (Rockville, Md.). The Dynabeads Antibody Coupling Kit was purchased from Thermo Fisher Scientific (Vilnius, Lithuania). Recombinant human target protein, rat anti-target monoclonal antibody, biotinylated sheep anti-target polyclonal antibody and horseradish peroxidase-conjugated streptavidin were purchased from R&D Systems (Minneapolis, Minn.). The soluble target receptor and co-factor proteins were purchased from Sigma (St Louis, Mo.). Black micro-well plates, horseradish peroxidase-conjugated NeutrAvidin and SuperSignal ELISA Pico chemiluminescent substrate were purchased from Thermo Fisher Scientific (Rockford, Ill.). MAB-Y is a fully human monoclonal antibody drug.

### Methods

### 1. Determination of pH

The determinations of pH were performed using a calibrated Mettler Toledo meter (Columbus, Ohio) with an InLab Expert Pro-ISM electrode. Pooled human serum was diluted 10-fold in 300 mM acetic acid. The acidified samples were then diluted 5-fold with different concentrations of Tris-base solutions with 50 μg/mL of the receptor and 50 μg/mL of the co-factor. The measurements of pH were performed on the final assay solutions as shown in Table 1.

TABLE 1
Evaluation of pH conditions for detecting
anti-MAB-Y ADA

| Solution | pH |
|---|---|
| 75 mM Tris | 7.4 |
| 60 mM Tris | 6.5 |
| 50 mM Tris | 6.0 |
| 40 mM Tris | 5.5 |
| 30 mM Tris | 5.0 |

2. Coupling of Magnetic Beads with Anti-Target Antibody

In order to deplete the target protein, for example, the target protein which can be recognized by an antibody drug, an anti-target antibody was coupled to magnetic beads for performing immuno-depletion. Anti-target antibody MAB-A was coupled to Dynabeads according to the manufacturer's instruction. Appropriate amounts of Dynabeads were washed with 1 mL of C1 solution from the kit and were then re-suspended with an appropriate volume of anti-target antibody MAB-A diluted in C1 solution. An equivalent volume of C2 solution was then added to the mixture followed by incubation at 37° C. for 16-24 hours. The coupled beads were sequentially washed with the HB, LB and SB buffers from the kit. The coupled beads were then re-suspended with SB buffer and were incubated at room temperature for approximately 15 minutes. The supernatant was removed. The MAB-A conjugated Dynabeads were re-suspended in SB buffer at a concentration of 10 mg/mL and stored at 4° C. until use.

### 3. Bridging ADA Assays

Immunoassays were developed to detect the presence of ADA in serum samples. The presence of ADA, such as anti-MAB-Y antibodies, in monkey and human serum samples were detected using a bridging immunoassay, for example, a bridging ADA assay. A mouse anti-drug monoclonal antibody, such as a mouse anti-MAB-Y antibody, was used as positive control. Biotinylated drug (Bio-drug) and ruthenium labeled drug (Ru-drug), such as biotinylated MAB-Y (Bio-MAB-Y) and ruthenium labeled MAB-Y (Ru-MAB-Y), were used as components to establish a bridge complex, for example, a bridge complex comprising ADA, Bio-drug and Ru-drug (for example, a bridge complex comprising anti-MAB-Y antibody, Bio-MAB-Y and Ru-MAB-Y).

Serum samples containing anti-MAB-Y antibodies were acidified using acetic acid prior to conducting the bridging ADA assay, such as conducting 10-fold dilution in 300 mM acetic acid with subsequent incubation at room temperature for at least 10 min. In order to achieve a bridging ADA assay which has a neutral pH, Bio-MAB-Y (1.0 μg/mL) and Ru-MAB-Y (1.0 μg/mL) were prepared in assay buffer containing 75 mM of Tris-base prior to incorporating them to the serum sample.

Acid-treated serum samples were diluted 5-fold in the labeled drug solution, for example, solution containing Bio-MAB-Y and/or Ru-MAB-Y, and subsequently the samples were incubated for approximately 60 min at room temperature. After incubation, samples were transferred to blocked (5% BSA) Streptavidin Multi-Array® 96-well plates (from MSD, i.e., Meso Scale Discovery, LLC) and incubated for approximately 60 min at room temperature. The plates were washed. Read Buffer was added to the plates for reading the plates using a MSD plate reader.

### 4. Total Drug Assays

Assay methods were developed to detect the presence of drugs in serum samples. In order to analyze the presence of drugs in monkey serum sample, for example, total amount of drugs in serum sample, a micro-titer plate was coated with a mouse anti-human IgG4 antibody (2 μg/mL). MAB-Y was used as a standard for the total drug assay. Acidification was used to dissociate the soluble target-drug complexes using acetic acid. Monkey serum samples, standards and controls were treated with 30 mM acetic acid to dissociate soluble target-drug complexes present in the serum samples. In addition, the acidification treatment was used to improve the detection of drugs in the presence of soluble targets in the serum. After capturing MAB-Y on the micro-titer plate, MAB-Y was detected using a biotinylated mouse anti-human Ig which was a kappa light chain specific monoclonal antibody (100 ng/mL) in combination with NeutrAvidin conjugated horseradish peroxidase (NeutrAvidin-HRP, 50 ng/mL). All incubations were performed at room temperature for approximately 60 min. Subsequently, a luminol-based substrate which was a peroxidase-specific substrate was used for generating detection signal. A signal intensity which was proportional to the concentrations of total MAB-Y was obtained. The plate was read on a microplate luminometer.

In order to analyze the presence of drugs in human serum sample, for example, total amount of drugs in serum sample, a micro-titer plate was coated with a mouse anti-MAB-Y monoclonal antibody (2 μg/mL). MAB-Y was used as a standard for the total drug assay. Acidification was used to dissociate the soluble target-drug complexes using acetic acid. Human serum samples were treated with 30 mM acetic acid to dissociate soluble target-drug complexes present in the serum samples. After capturing MAB-Y on the micro-titer plate, MAB-Y was detected using a different biotinylated mouse anti-MAB-Y specific monoclonal antibody (100 ng/mL) in combination with NeutrAvidin conjugated horseradish peroxidase (NeutrAvidin-HRP, 50 ng/mL). All incubations were performed at room temperature for approximately 60 min. Subsequently, a luminol-based substrate which was a peroxidase-specific substrate was used for generating detection signal. A signal intensity which was proportional to the concentrations of total MAB-Y was obtained. The plate was read on a microplate luminometer.

### 5. Total Target Assays

Assay methods were developed to detect the presence of target proteins in serum samples. In order to analyze the presence of target proteins in the serum samples, for example, total amount of target proteins in serum sample, a micro-titer plate was coated with a rat anti-target monoclonal antibody (4 μg/mL). A recombinant target protein was used as standard. Acidification was used to dissociate the soluble target-drug complexes using acetic acid. Serum samples, standards and controls were diluted at the ratio of 1:10 in 300 mM acetic acid to dissociate soluble target-drug complexes that might be present in serum samples, which was followed by neutralization with a 1:5 dilution in a 75 mM Tris solution. Neutralized standards, controls and samples were then added to the micro-titer plate. The target proteins which were captured on the plate were detected with a biotinylated sheep anti-target polyclonal antibody (100 ng/mL) in combination with streptavidin conjugated horseradish-peroxidase (streptavidin-HRP, 1:200 dilution in ADB). All incubations were performed at room temperature for approximately 60 min. Subsequently, a luminol-based substrate which was a specific substrate for peroxidase was added for generating a detection signal. A signal intensity which was proportional to the concentrations of total targets was obtained. The plate was read on a microplate luminometer.

### 6. Immuno-Depletion of Target Proteins

Two methods, for example, methods A and B, were developed for immuno-depletion of target proteins in serum samples. In method A of immuno-depletion of target proteins, serum samples were diluted 10-fold in 300 mM acetic acid and were incubated at room temperature for at least 10 minutes. The acidified samples were then neutralized with a 1:3 dilution in a 150 mM Tris solution. Magnetic beads conjugated with the anti-target antibody MAB-A were washed once with 1×PBS and were then re-suspended with the neutralized serum samples. After incubation at room temperature for approximately 60 min, the supernatant was collected and were then mixed with a solution containing 2 μg/mL of Bio-MAB-Y and 2 μg/mL of Ru-MAB-Y. After incubation at room temperature for approximately 60 min, samples were transferred to blocked (5% BSA) Streptavidin Multi-Array® 96-well plates (from MSD) and further incubated for approximately 60 min at room temperature. The plates were washed, Read Buffer was added. The plates were read using a MSD plate reader.

In method B of immuno-depletion of target proteins, serum samples were diluted 10-fold in 30 mM acetic acid and were then incubated with magnetic beads which were conjugated with the anti-target antibody MAB-A for approximately 30 min at room temperature. The supernatant was collected and then diluted 3-fold in a 10 mM Tris solution containing 1 μg/mL of Bio-MAB-Y and 1 μg/mL of Ru-MAB-Y. After incubation at room temperature for approximately 60 min, samples were transferred to blocked (5% BSA) Streptavidin Multi-Array® 96-well plates (from MSD) and were further incubated for approximately 60 min at room temperature. The plates were washed, Read Buffer was added. The plates were read using a MSD plate reader.

### Example 1. The Use of Anti-Target Antibodies to Improve ADA Detection

When a drug target is a multimeric protein which is present in serum, the drug target can generate target-mediated false-positive signal which can interfere ADA detection. ADAs of MAB-Y in serum samples can be detected using Ru-MAB-Y and Bio-MAB-Y by forming a complex comprising Ru-MAB-Y, ADA and Bio-MAB-Y, for example, using ADA to bridge Ru-MAB-Y and Bio-MAB-Y. However, since the target of MAB-Y (e.g., drug) is a multimeric protein which is expressed at different levels in monkey and human naïve serum samples, the target in serum can form a complex with Ru-MAB-Y and Bio-MAB-Y, for example, using target to bridge Ru-MAB-Y and Bio-MAB-Y, which contribute to target-mediated false-positive signal. Anti-target antibodies were used to mitigate the interference of false-positive signal caused by the bridging effects of the drug target.

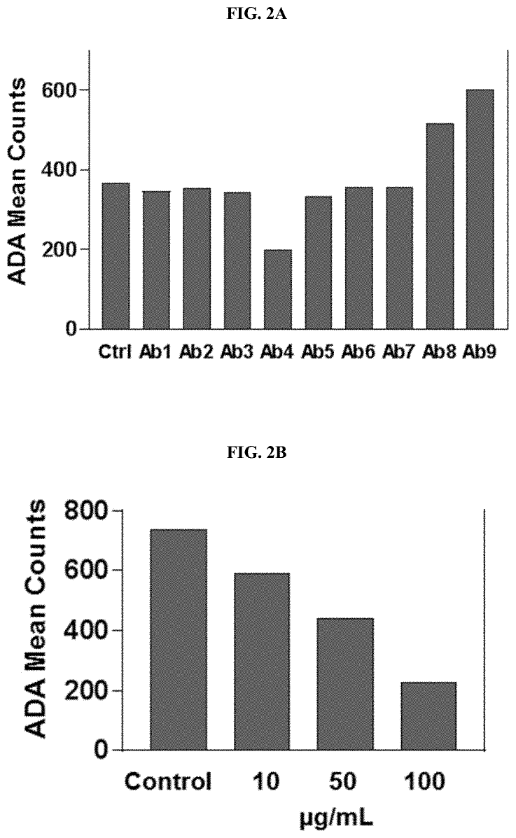

Anti-target antibodies are frequently used to mitigate target interference in bridging ADA assays (Liao, et al., Inhibition of interleukin-5 induced false positive anti-drug antibody responses against mepolizumab through the use of a competitive blocking antibody. J Immunol Methods, 2017. 441: p. 15-23; Zhong, et al., Identification and inhibition of drug target interference in immunogenicity assays. J Immunol Methods, 2010. 355(1-2): p. 21-8; Dai, et al.; Weeraratne, et al., Development of a biosensor-based immunogenicity assay capable of blocking soluble drug target interference. J Immunol Methods, 2013. 396(1-2): p. 44-55; Maria, et al., A novel strategy for elimination of soluble-ligand interference in immunogenicity assays. AAPS National Biotechnology Conference. Seattle, Wash., USA, 2009). Several anti-target antibodies, for example, Ab1-Ab9, at 100 μg/mL were screened in comparing to control (Ctrl) for mitigating target interferences in monkey naïve serum sample as shown in FIG. 2A. Among the anti-target antibodies screened, only one anti-target antibody, for example, Ab4, was able to partially inhibit target-mediated false-positive signal as shown in FIG. 2A. Two of the tested antibodies, for example, Ab8 and Ab9, actually potentiated (increased) the target-mediated false-positive signal. Various combinations of these antibodies were also evaluated but they failed to sufficiently inhibit target interference in monkey serum samples (data not shown).

Several rounds of immunization were further performed to generate more anti-target antibodies. However, none of the screened anti-target antibodies can adequately compete with MAB-Y. One commercially available polyclonal anti-target antibody was used to mitigate target interference. This polyclonal anti-target antibody exhibited certain effects to mitigate the target interference in a dosage-dependent manner as shown in FIG. 2B. However, polyclonal antibodies can exhibit batch-to-batch variability which can have negative impacts on assay development regarding target interference mitigation.

### Example 2. The Use of Target Receptor to Improve ADA Detection

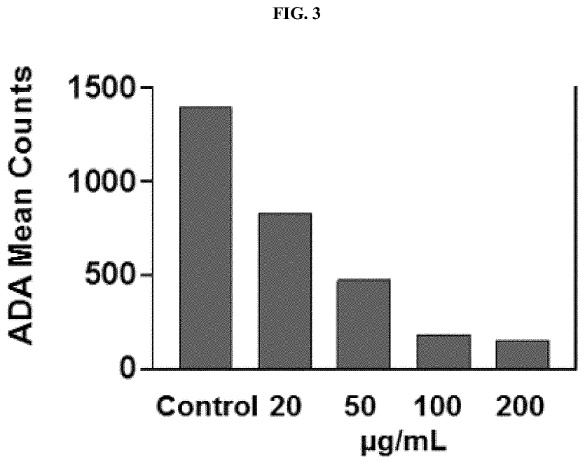

Target receptor was incorporated to the bridging ADA assay to improve ADA quantitation by mitigating target-mediated signals. A soluble target receptor (such as 50 μg/mL) was included in the labeled drug solution. The labeled drug solution was prepared in a 50 mM Tris solution to adjust the assay pH to a mild acidic condition at about pH 6.0. The mild acidic condition can also minimize the binding of the target to both Bio-MAB-Y and Ru-MAB-Y. The results indicated that the addition of soluble target receptor can significantly reduce the target-mediated signal in naïve monkey serum samples. The soluble target receptor was able to mitigate target-mediated signal in a dosage-dependent manner as shown in FIG. 3. Y axis indicates ADA mean counts and X axis indicates the concentrations of target receptor in μg/mL in FIG. 3. The soluble target receptor effectively blocked the target interference in a naïve monkey serum sample at 100 μg/mL.

### Example 3. The Use of Target Receptor and Co-Factor to Improve ADA Detection

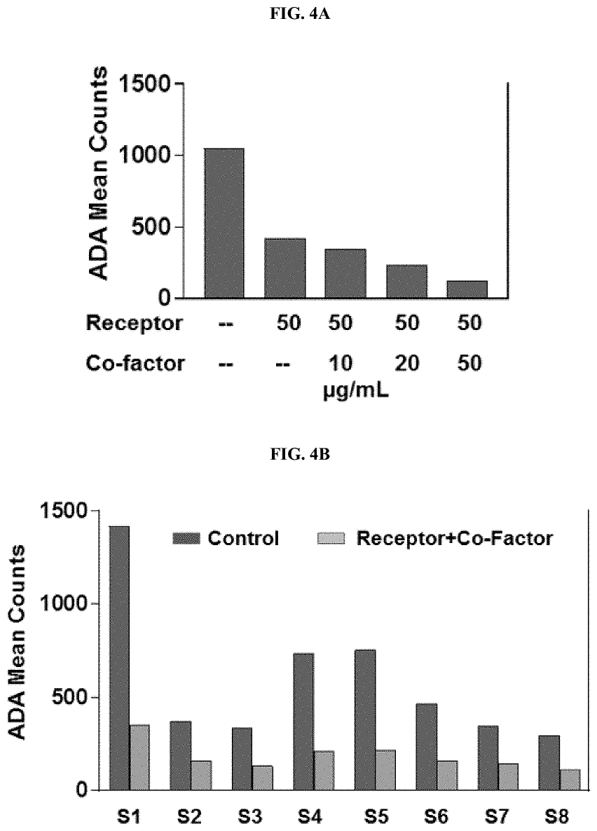

Target receptor and co-factor were incorporated to the bridging ADA assay to improve ADA detection by mitigating target-mediated signals. A soluble target receptor (such as 50 μg/mL) and co-factor protein (such as 50 μg/mL) were included in the labeled drug solution. The labeled drug solution was prepared in a 50 mM Tris solution to adjust the assay pH to a mild acidic condition at about pH 6.0. The mild acidic condition can also minimize the binding of the target to both Bio-MAB-Y and Ru-MAB-Y. Different concentrations of the co-factor protein were added to the solution containing 50 μg/mL of the soluble target receptor for conducting bridging ADA assay as shown in FIG. 4A. Y axis indicates ADA mean counts and X axis indicates the concentrations of target receptor and/or co-factor in μg/mL in FIG. 4A. The results indicated that the combination of the soluble target receptor and co-factor proteins can significantly reduce the target-mediated signal in naïve monkey serum samples. The two proteins, for example, target receptor and co-factor, functioned together synergistically to effectively reduce the background signal in a monkey naïve sample. The results indicated that the combination of 50 μg/mL of the receptor and 50 μg/mL of the co-factor was most effective as shown in FIG. 4A.

A widely variable range of target-mediated assay signals were detected in the absence of any blocker proteins in eight naïve monkey serum samples as shown in FIG. 4B (control), which may likely reflect natural variability in endogenous target levels. The presence of target receptor and co-factor, for example, the combination of 50 μg/mL of the receptor and 50 μg/mL of the co-factor, showed effective mitigation of the target-mediated assay signals in all monkey serum samples as shown in FIG. 4B. However, one serum sample still had a signal of approximately 400 Mean Counts.

### Example 4. Optimizing Assay pH to Mitigate Target Interference

MAB-Y binding affinity to its target was greatly reduced under acidic conditions (about pH 6.0), when compared to neutral pH. To test whether mild acidic assay pH can inhibit target-mediated signal in the bridging ADA assay, four experimental designs were conducted for optimizing assay pH. The four experimental designs were (1) four monkey naïve serum samples alone without any blocker at neutral pH (control); (2) four monkey naïve serum samples with 50 μg/mL of the receptor and 50 μg/mL of the co-factor at neutral pH; (3) four monkey naïve serum samples alone without any blocker at mild acidic pH at about pH 6.0; and (4) four monkey naïve serum samples with 50 μg/mL of the receptor and 50 μg/mL of the co-factor at mild acidic pH at about pH 6.0.

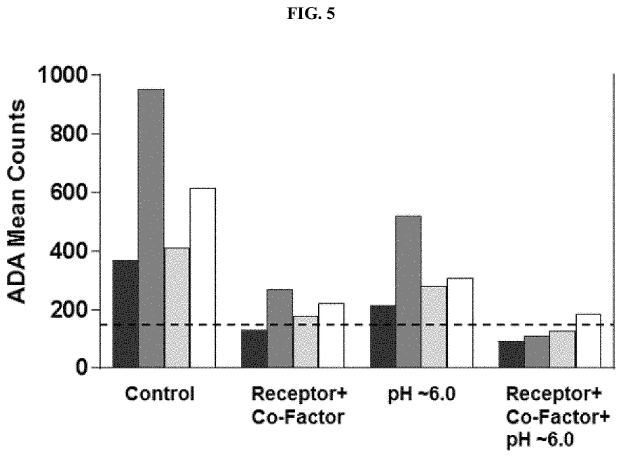

The results indicated that the high background signal from naïve monkey serum samples was partially inhibited by mild acidic pH alone (pH ˜6.0) as shown in FIG. 5. The results of pH optimization indicated that mild acidic assay pH can inhibit target interference in monkey serum samples. The combination of the receptor and the co-factor proteins was able to significantly reduce the target-mediated signal at both pH conditions, for example, at neutral pH and at mild acidic pH. In particular, the combination of receptor, co-factor and mild acidic assay conditions (pH ˜6.0) provided synergistic effects to completely inhibit target-mediated signal as shown in FIG. 5.

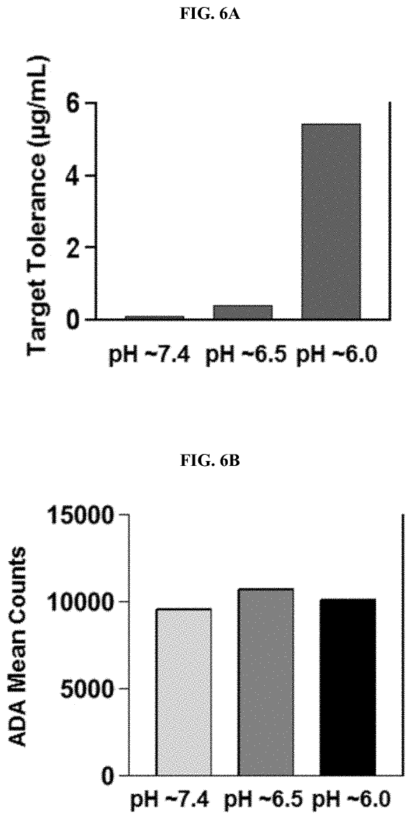

The target tolerance level was determined using a recombinant target protein under different assay pH conditions, when the ADA assay was performed using 50 μg/mL of both the receptor and co-factor proteins. The target tolerance level was defined as the amount of target needed to obtain an assay signal above the plate cut point. The target tolerance level was determined to be approximately 94 ng/mL, when the ADA assay was performed using 50 μg/mL of both the receptor and co-factor proteins under the neutral assay pH conditions as shown in FIG. 6A. The target tolerance level increased to approximately 380 ng/mL with the same concentration of the receptor and co-factor, when the assay pH was around 6.5. The target tolerance level was even higher, at approximately 5.0 μg/mL, when the assay pH was around 6.0. The results indicated that a mild acidic assay pH can significantly improve the mitigation of target-mediated signals.

To ensure that the mild acidic pH has minimal impact on the stability and/or the detection of a true ADA signal, early bleeds from MAB-Y Fab-immunized rabbits were analyzed at different assay pH conditions. Bleed 1 was collected approximately 30 days after immunization, and the induced antibody response in this bleed was typically comprised of low affinity polyclonal ADA, the detection of which may be more impacted by stringent assay conditions. As shown in FIG. 6B, the ADA mean count values were similar at each assay condition regardless of the assay pH conditions. The results indicated that the mild acidic pH had minimal to no impact on the stability and/or the detection of true ADA in these samples. The results indicated that mild acidic assay pH improved target tolerance levels and had minimal impact on true ADA detection.

### Example 5. The Combination of Target Receptor, Co-Factor and Mild Acidic Assay pH

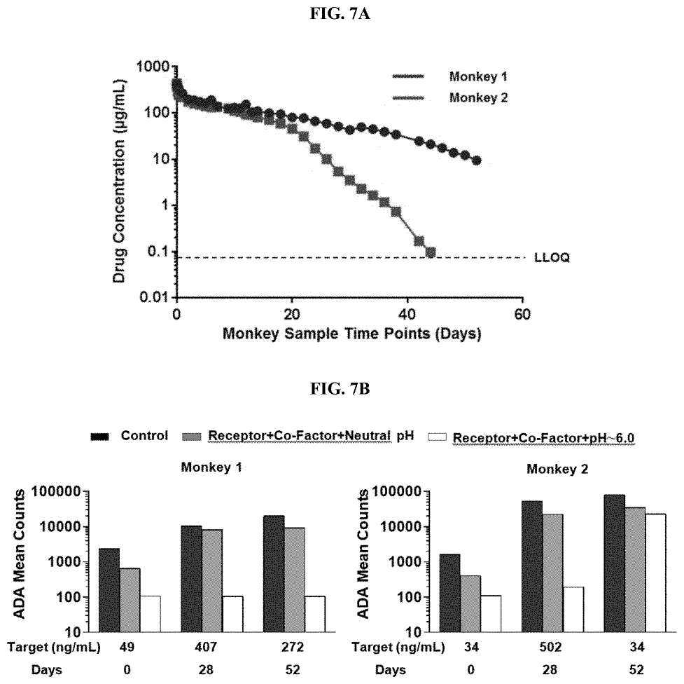

The combination of target receptor, co-factor and mild acidic assay pH was incorporated to the bridging ADA assay to improve ADA detection by mitigating target-mediated signals. Monkey post-dose samples which usually contain higher levels of target protein were used for conducting the bridging ADA assays. Serum samples from two monkeys on days 0, 28 and 52 after a single dose of drug (e.g. MAB-Y) were tested using the bridging ADA assay. The drug concentrations in serum samples indicated drug pharmacokinetics (PK) profiles of these two monkeys are shown in FIG. 7A (LLOQ indicates lower limit of quantitation). Monkey 1 exhibited a linear PK profile. Monkey 2 showed a significant decrease in drug levels, for example, accelerated drug clearance, starting from Day 21, which could indicate a significant ADA response in Monkey 2.

The concentrations of target and ADA in the monkey serum samples were measured. FIG. 7B shows target concentrations and ADA signals in Day 0, 28 and 52 samples with different assay conditions using monkey post-dose samples according to an exemplary embodiment. Compared to baseline, target levels increased approximately 10 to 15 fold in Day 28 samples from both monkeys, and the target concentration remained high in Day 52 sample from Monkey 1. When the bridging ADA assay was performed using the post-dose samples under control conditions without the presence of any competitive blockers at a neutral assay pH, a strong assay signal was obtained from these serum samples as shown in FIG. 7B. The addition of the receptor and co-factor molecules in bridging ADA assays at a neutral assay pH partially inhibited the assay signal in these samples. The baseline samples showed a more noticeable reduction in signal. However, since the ADA Mean Counts remained far above the plate cut point for all samples under these conditions, it was difficult to distinguish a true ADA signal from the target-mediated false positive signal.

When the monkey serum samples were tested in bridging ADA assay in the presence of 50 μg/mL of receptor and 50 μg/mL of co-factor under mild acidic assay pH (e.g. about pH 6.0), a low background signal was detected for all samples from Monkey 1 as shown in FIG. 7B. For Monkey 2, approximately 2-fold and 200-fold increase in the ADA signal were observed for the Day 28 and Day 52 samples, respectively, compare to baseline as shown in FIG. 7B. The results indicated that the combination of the soluble receptor, co-factor and mild acidic assay pH can mitigate target interference and detect true ADAs in monkey post-dose samples. The results also indicated that the serum samples of Monkey 1 had no ADA and that the serum samples of Monkey 2 had ADA responses, which were supportive to the drug concentration profiles of these two monkeys as shown in FIG. 7A.

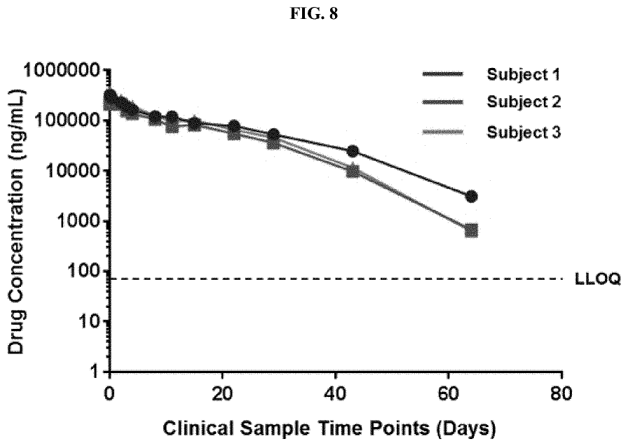

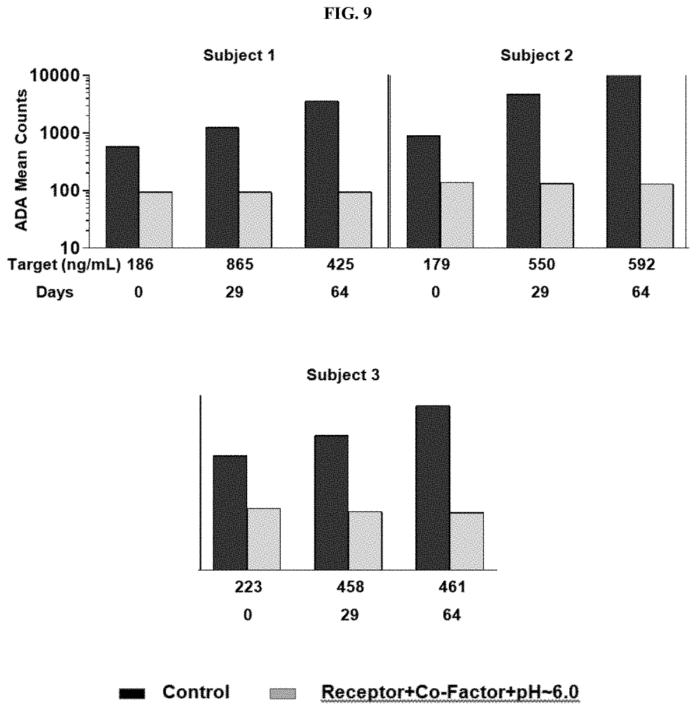

Clinical study samples (Day 0, 29 and 64) from three subjects from a phase I clinical study were also tested using bridging ADA assay by incorporating the combination of target receptor, co-factor and mild acidic assay pH to improve ADA detection by mitigating target-mediated signals. The drug concentrations in these clinical study samples were measured. The drug concentrations in serum samples of three subjects with a single dose of MAB-Y were measured as shown in FIG. 8. LLOQ indicates lower limit of quantitation. The PK profiles of these samples did not suggest significant ADA responses. However, high assay signal was observed for all samples as shown in FIG. 9, when these samples were tested in the bridging ADA assay without the presence of any blocker molecules under neutral assay pH. FIG. 9 shows target concentrations and ADA signals in Day 0, 29 and 64 samples with different assay conditions including the incorporation of target receptor, co-factor and mild acidic assay pH to bridging ADA assay to improve ADA detection according to an exemplary embodiment.

These assay signals appeared to correlate with the target levels in these samples. Target concentrations increased in the post-dose samples in all three subjects. High target-mediated signals were detected in all samples without the presence of the blockers under neutral assay pH. When these clinical study samples were tested again in the presence of the soluble receptor (50 μg/mL) and co-factor (50 μg/mL) under mild acidic assay pH (about pH 6.0), only background signal was detected as shown in FIG. 9. A large set of clinical study samples (Day 0, 29 and 64 samples from 11 subjects) was subsequently tested and all samples demonstrated only background signal (data not shown). The results indicated that the assay format of incorporating two competitive blocker proteins (e.g., target receptor and co-factor) under mild acidic assay conditions can effectively mitigate target interference in human post-dose serum samples. The results were also supportive to the PK profiles which did not suggest a positive ADA response.

### Example 6. Immuno-Depletion of Target Proteins Under Neutral Assay pH

Immuno-depletion has been used to remove various target proteins from serum samples to inhibit the target-mediated assay signal (Dai, et al.). A similar approach was explored to remove the multimeric target protein from human serum samples. MAB-A, an anti-target antibody, was used for conducting immuno-depletion, which was not able to compete with drug MAB-Y for target binding at neutral assay pH. Instead, MAB-A may potentiate target-mediated signal in human serum samples (data not shown).

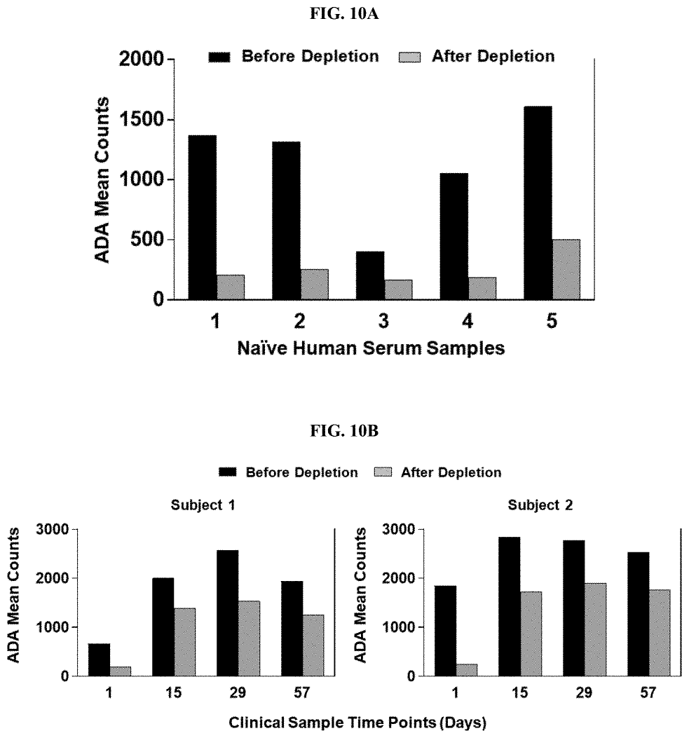

A different approach was conducted using MAB-A conjugated magnetic beads for immuno-depletion, which was able to successfully remove target protein from five human naïve serum samples thereby inhibiting the target-mediated signal as shown in FIG. 10A. Target-mediated signals were eliminated in drug-free naïve human serum samples by immuno-depletion of the target protein with MAB-A conjugated magnetic beads at neutral assay pH. However, one sample still demonstrated a signal of approximately 450 Mean Counts after target removal. The MAB-A conjugated magnetic beads were also used to remove the target protein from clinical study samples, for example, Day 1, 15, 29 and 57 samples from two subjects with a single dose of MAB-Y. The results indicated that only baseline samples demonstrated a reduction to background signal. A significant level of target-mediated signal was still detected in the post-dose samples as shown in FIG. 10B. These post-dose samples contained high concentrations of drug MAB-Y. Since MAB-A does not compete with MAB-Y for target binding at the neutral assay pH, the anti-target antibody-coupled beads may not be able to fully deplete the target protein from the serum samples in the presence of high concentrations of MAB-Y. In the post-dose samples, when the drug was still present at a high concentration, target-mediated assay signal was not completely inhibited. The results indicated that immuno-depletion with MAB-A conjugated magnetic beads under neutral assay pH conditions was not sufficient to mitigate target interference in human post-dose samples.

### Example 7. Immuno-Depletion of Target Proteins Under Mild Acidic Assay pH

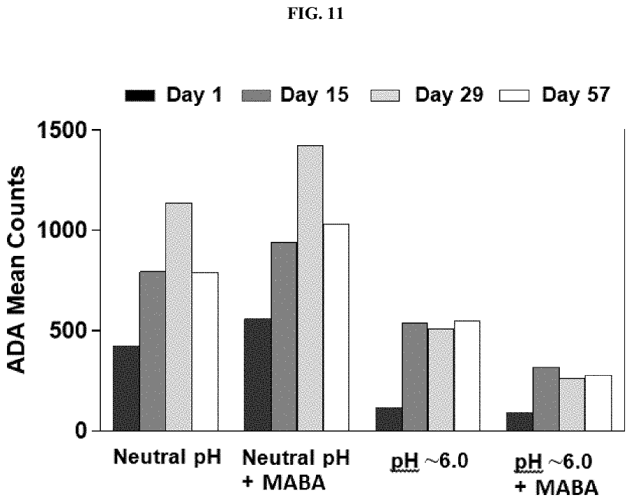

Anti-target antibody (MAB-A) and drug MAB-Y exhibit similar K_{D} value at the neutral assay pH, although the t1/2 of MAB-A is slightly greater than that of MAB-Y as shown in Table 2. However, at pH ˜6.0, the anti-target antibody demonstrates much better binding to the target, with a far longer t1/2 (Table 2). To test whether MAB-A can compete with MAB-Y for target binding at a mild acidic pH (pH ˜6.0), Day 1, 15, 29 and 57 clinical study samples were tested with or without MAB-A at either neutral or mild acidic assay pH (pH ˜6.0). At neutral assay pH, the addition of MAB-A failed to inhibit target-mediated signal. Instead, the antibody slightly potentiated the observed target interference in the tested samples as shown in FIG. 11. With mild acidic pH alone, the assay signal decreased, especially in the baseline samples. When MAB-A was added to the mild acidic assay solution, inhibition of target-mediated signal was observed for the post-dose samples, even for samples in which MAB-Y was still present at high concentrations as shown in FIG. 11. FIG. 11 shows ADA assay signal in Day 1, 15, 29 and 57 samples without blockers under neutral assay pH, with 100 μg/mL MAB-A under neutral assay pH, without blockers under mild acidic pH (pH ˜6.0), and with 100 μg/mL MAB-A under mild acidic pH (pH ˜6.0). The results indicated that MAB-A can compete with MAB-Y when the assay pH was mild acidic.

TABLE 2
K_{D} and t½ values of MAB-Y and MAB-A.

| | pH ~7.0 25° C. | | pH ~6.0 25° C. | |
|---|---|---|---|---|
| Antibody | K_{D} (M) | t½ (min) | Kd (M) | t½ |
| MAB-Y | 6.70E−10 | 28.8 | 6.75E−08 | 2.3 |
| MAB-A | 6.80E−10 | 62.1 | 6.49E−10 | 48.2 |

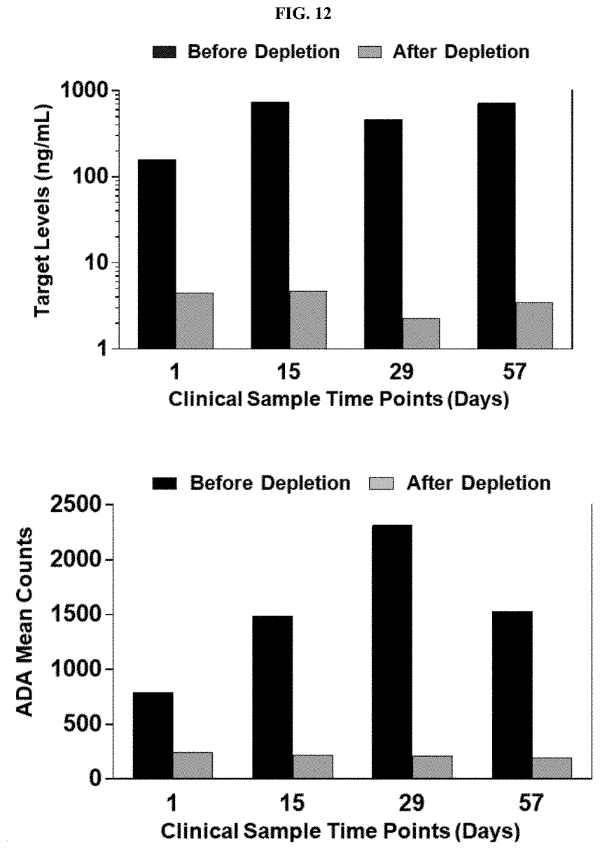

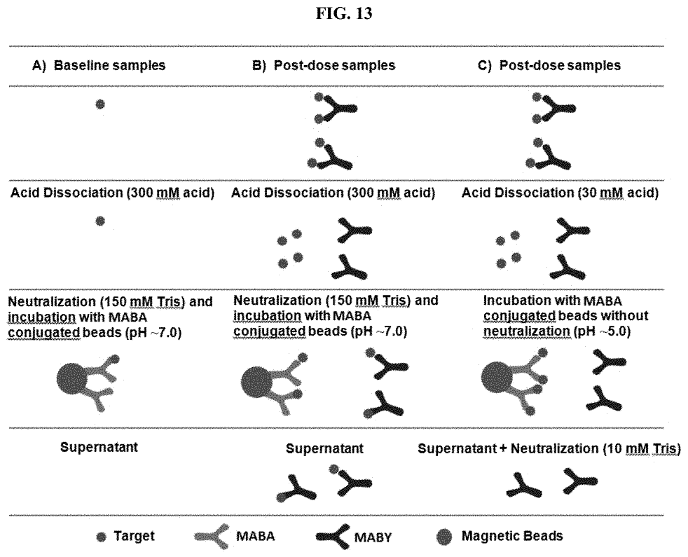

Relative to the baseline samples, the target concentration in Day 15, 29 and 57 samples increased approximately 3- to 5-fold before returning to baseline levels at Day 183 as shown in FIG. 12. FIG. 12 shows target concentrations and ADA assay signal in Day 1, 15, 29, and 57 samples before and after immuno-depletion with MAB-A conjugated magnetic beads under mild acidic assay pH according to an exemplary embodiment. To ensure that the modified immuno-depletion method was able to deplete target proteins in these samples, target concentrations were measured before and after conducting immuno-depletion. Before conducting immuno-depletion, the target levels ranged from 150 ng/mL to 750 ng/mL, whereas the target concentrations were only about 2 to 5 ng/mL after immuno-depletion. The results indicated that the target protein was efficiently removed as shown in FIG. 12. When clinical study samples were tested in the modified immune-depletion ADA assay, only background signal (approximately 200 Mean Counts) was detected, compared to the Mean Counts of 1500 to 2300 observed without immuno-depletion as shown in FIG. 12. These results indicated that MAB-A can effectively compete with MAB-Y for target binding when the assay pH was about 5.0 and therefore was able to completely deplete target protein from the analyzed clinical study samples as shown in FIG. 13. Immuno-depletion of multimeric target protein from clinical study samples with MAB-A conjugated magnetic beads were conducted under mild acidic assay pH.

The results indicated that immuno-depletion with MAB-A conjugated magnetic beads under mild acidic assay conditions can mitigate target interference in human post-dose samples. As shown in FIG. 13, for baseline samples, MAB-A conjugated magnetic beads was able to effectively remove the target protein. For post-dose samples, even though MAB-Y was present at a high concentration, acid treatment with 300 mM acetic acid was able to dissociate the target-drug complexes. After neutralization and the addition of MAB-A conjugated magnetic beads, some of the target protein was able to bind to MAB-A on the beads and some of them was able to re-associate with MAB-Y, since MAB-A was unable to compete with MAB-Y under neutral assay pH. Therefore, in the supernatant, there were still sufficient amount of re-formed target-drug complexes which was able to generate target-mediated signal. For the same post-dose samples, acid treatment with 30 mM acetic acid was able to dissociate the target-drug complexes. When the acidified samples were incubated with the MAB-A conjugated magnetic beads under this acidic condition, the free target protein was able to preferably bind to MAB-A on the beads, and not to MAB-Y in the supernatant, since MAB-A had a much better affinity for target binding when the assay pH was between 5 and 6 and with a much greater t1/2, compare to MAB-Y. Therefore, only unbound REGN-Y was present in the supernatant.

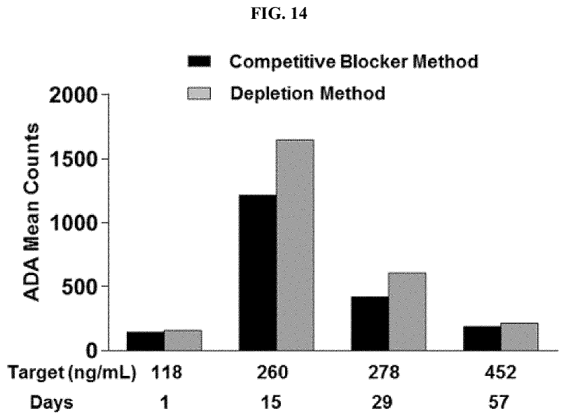

Day 1, 15, 29 and 57 samples from an ADA-positive subject identified using the competitive blocker ADA method, e.g, with competitive blocking from the soluble receptor (50 μg/mL) and co-factor (50 μg/mL) under mild acidic pH, were tested. With the competitive blocker ADA method, the ADA signal was found to increase by approximately 8-fold for the Day 15 sample, compared to the Day 1 sample, and approximately 3-fold for the Day 29 sample as shown in FIG. 14. With the immune-depletion method, similar increases in the ADA signal were observed for the Day 15 and Day 29 samples. The results indicated that this method also enabled detection of a true ADA signal as shown in FIG. 14. For the Day 57 sample, even though the serum target concentration was approximately 450 ng/mL, the assay signal remained at background levels. The results also indicated that both methods can successfully inhibit the target interference signals. The results indicated that modified immune-depletion method can detect true ADA responses.

What is claimed is:

1. A method of identifying an anti-drug antibody in a sample, comprising:
contacting the sample with a first labeled drug,
contacting the sample with a second labeled drug,
contacting the sample with a binding partner of a target, and
detecting the presence of a complex which comprises the first labeled drug, the anti-drug antibody and the second labeled drug;
wherein the sample comprises the anti-drug antibody and the target, and
wherein the target is a binding partner of the drug.

2. The method of claim 1 further comprising contacting the sample with a co-factor to enhance the binding between the target and the binding partner of the target.

3. The method of claim 1, wherein the method is conducted under a mild acidic assay pH.

4. The method of claim 1 further comprising removing the target using an anti-target antibody.

5. The method of claim 4, wherein the anti-target antibody is attached to a solid support.

6. The method of claim 1, wherein the first labeled drug is ruthenium labeled drug or biotinylated drug.

7. The method of claim 1, wherein the second labeled drug is ruthenium labeled drug or biotinylated drug.

8. The method of claim 1, wherein the binding partner of the target is a natural binding partner.

9. The method of claim 1, wherein the binding partner of the target is a receptor of the target.

10. The method of claim 1, wherein the target is a soluble multimeric target.

11. The method of claim 3, wherein the mild acidic assay pH is in the range of from about pH 4.5 to about pH 6.5.

12. The method of claim 3, wherein the mild acidic assay pH is about pH 6.0.

13. The method of claim 3, wherein the mild acidic assay pH is about pH 5.0.

14. The method of claim 1, wherein the drug is a chemical compound, a nucleic acid, a toxin, a peptide, a protein, a fusion protein, an antibody, an antibody fragment, a Fab region of an antibody, an antibody-drug conjugate, or a pharmaceutical product.

15. The method of claim 1, wherein the drug is an antibody.

16. The method of claim 1, wherein the sample is a serum sample.

17. A system for identifying an anti-drug antibody in a sample, comprising:
a first labeled drug,
a second labeled drug,
a binding partner of a target, and
an assay system to detect the presence of a complex which comprises the first labeled drug, the anti-drug antibody and the second labeled drug;
wherein the sample comprises the anti-drug antibody and the target, and
wherein the target is a binding partner of the drug.

18. The system of claim 17 further comprising a co-factor which can enhance the binding between the target and the binding partner of the target.

19. The system of claim 17, wherein the sample is treated with a solution having a mild acidic assay pH.

20. The system of claim 17 further comprising an anti-target antibody.

21. The system of claim 20, wherein the anti-target antibody is attached to a solid support.

22. The system of claim 17, wherein the first labeled drug is ruthenium labeled drug or biotinylated drug.

23. The system of claim 17, wherein the second labeled drug is ruthenium labeled drug or biotinylated drug.

24. The system of claim 17, wherein the binding partner of the target is a natural binding partner.

25. The system of claim 17, wherein the binding partner of the target is a receptor of the target.

26. The system of claim 17, wherein the target is a soluble multimeric target.

27. The system of claim 19, wherein the mild acidic assay pH is in the range of from about pH 4.5 to about pH 6.5.

28. The system of claim 19, wherein the mild acidic assay pH is about pH 6.0.

29. The system of claim 19, wherein the mild acidic assay pH is about pH 5.0.

30. The system of claim 17, wherein the drug is a chemical compound, a nucleic acid, a toxin, a peptide, a protein, a fusion protein, an antibody, an antibody fragment, a Fab region of an antibody, an antibody-drug conjugate, or a pharmaceutical product.

31. The system of claim 1, wherein the drug is an antibody.

32. The system of claim 1, wherein the sample is a serum sample.
